(19) United States
(12) Patent Application Publication
(10) Pub. No.: US 2003/0049689 A1
(43) Pub. Date: Mar. 13, 2003

(54) Multifunctional polypeptides

(76) Inventors: Cynthia Edwards, Redwood City, CA (US); Volker Schellenberger, Palo Alto, CA (US); Christopher J. Murray, Soquel, CA (US); Yiyou Chen, San Jose, CA (US); Anthony G. Day, San Francisco, CA (US)

Correspondence Address: PENNIE AND EDMONDS, 1155 AVENUE OF THE AMERICAS, NEW YORK, NY, 100362711

(21) Appl. No.: 10/170,729

(22) Filed: Jun. 12, 2002

Related U.S. Application Data
(63) Continuation-in-part of application No. 10/022,073, filed on Dec. 13, 2001.
(63) Continuation-in-part of application No. 10/022,097, filed on Dec. 13, 2001.
(60) Provisional application No. 60/255,774, filed on Dec. 14, 2000.
(60) Provisional application No. 60/279,609, filed on Mar. 28, 2001.
(60) Provisional application No. 60/348,570, filed on Oct. 26, 2001.

(51) Int. Cl. G01N 33/53; G01N 33/567; G01N 33/574; C12P 21/02; C12N 5/06

(52) U.S. Cl. 435/7.1; 435/7.2; 435/69.1; 435/325; 435/7.23

(57) ABSTRACT

The present invention provides methods and compositions relating to polypeptides engineered to bind a target and have at least two functional domains.

### CROSS-REFERENCES TO RELATED APPLICATIONS

This application is a continuation-in-part of U.S. patent application Ser. Nos. 10/022,073 and 10/022,097, both filed Dec. 13, 2001, which claim priority under 35 U.S.C. §119(e) to U.S. Provisional Application Nos. 60/255,774, filed Dec. 14, 2000, 60/279,609, filed Mar. 28, 2001 and 60/348,570, filed Oct. 26, 2001, which are incorporated herein by reference in their entireties.

### FIELD OF THE INVENTION

The present invention provides methods and compositions relating to polypeptides engineered to bind a target and have at least two functional domains.

### BACKGROUND

The complexity of activities and responses mediated by proteins in biological organisms is remarkable, especially when considering that the number of proteins encoded in their genomes is comparatively small. This feat of complexity in function is accomplished by combinatorial relationships between individual proteins, each of which has relatively simplistic functional characteristics. That is, most naturally-occurring proteins have no more than two or three different types of functional domains that facilitate interacting with other proteins or other molecules (e.g., binding sites for proteins, DNA, or other macromolecules or ligands; catalytic sites; or recognition sites for modifications such as phosphorylation or glycosylation). The actual complexity of biological functions is then accomplished by the multitude of interactions between a relatively modest number of functionally simplistic proteins to yield a wealth of different pathways and networks, enabling biological diversity in both development and response. For example, several reports discuss the complexity of interactions between proteins in yeast. See, e.g., Gerstein et al., 2002, Science 295:284-87; Zhu et al., 2001, Science 293:2101-05; Tong et al., 2002, Science 295:321-24.

The number of functional domains in a protein—either engineered or naturally-occurring—naturally limits the specified activities of known proteins. For example, both naturally-occurring and engineered antibodies have been targeted to cancer cells for therapeutic or diagnostic purposes. These antibodies may have more than one (but usually not more than two) functional domains (independent of glycosylation sites): they have variable regions (CDRs), which are domains that confer the function of antigen-specific binding; in addition, they may be tagged with a radionuclide for the function of either imaging or toxicity; or covalently linked to a toxin; or they may be fused to an enzyme to confer the ability to convert a precursor molecule into an active molecule for the function of either diagnostics (e.g., in the case of a fluorogenic or colorimetric precursor) or therapeutics (e.g., in the case of prodrug to toxin conversion). The success of these molecules has been limited, often because they are not sufficiently specific in binding and the binding to normal tissue causes unwanted side effects. In addition, virtually all molecules that have been targeted to surfaces (e.g., tumors) are intended to exert a single activity (e.g., toxicity, detection, etc.) mediated by a single functional mechanism in addition to target binding.

Thus, there is a need in the art for polypeptides that more specifically bind a target and/or perform more than one function.

### SUMMARY OF THE INVENTION

The present invention provides methods and compositions relating to multifunctional polypeptides. The multifunctional polypeptides of the invention comprise at least three functional domains, at least one of which is a binding domain. The multifunctional polypeptides of the present invention are distinctly different from previously described polypeptides in that they are engineered to contain multiple functional domains, one or more of which confers a specific, targeted binding activity of the polypeptide to a target, and one or more of which confers a functional activity. An example of such a protein is one that is targeted via antigen-specific binding to a tumor with functional domains that act to kill the tumor cells by at least two separate mechanisms. Another example is a tumor-targeted protein that is significantly, e.g., an order of magnitude or more, more specific in binding because of a second, binding domain that drives higher avidity, selectivity or affinity, such that the tumor-targeted polypeptide can kill tumor cells without serious side effects to normal tissues.

In one aspect, the present invention provides a multifunctional polypeptide comprising a targeted peptide and at least two functional domains.

In one embodiment, the multifunctional polypeptide comprises a catalytic, activating, inhibiting, cytoxic, detectable or immunogenic functional domain.

In another embodiment, the targeted peptide binds to an enzyme, a serum protein, a receptor, a membrane-bound protein, a membrane or a tumor antigen. In another embodiment, the targeted peptide binds to a tissue, an organ or a cell. In another embodiment, the tissue, organ or cell is diseased, injured, infected or cancerous.

In another aspect, the present invention provides a method of making a multifunctional polypeptide comprising

a) selecting, from a library of peptides, a peptide comprising an amino acid sequence that binds to a target, and

b) making a multifunctional polypeptide that comprises the target-binding amino acid sequence of said peptide selected in step (a) and at least two functional domains.

In another aspect, the present invention provides a method of selectively targeting a tissue, organ or cell type in a subject comprising administering to said subject a multifunctional polypeptide, wherein said multifunctional polypeptide comprises a first low affinity binding domain for a first microtarget and a second low affinity binding domain for a second microtarget, the target tissue, organ or cell type comprises the first and the second microtargets, and a non-target tissue, organ or cell type does not comprise both the first and the second microtargets, such that the affinity and specificity of the multifunctional polypeptide is higher for the target tissue, organ or cell type than it is for the non-target tissue, organ or cell type. In one embodiment, the multifunctional polypeptide further comprises a functional domain. In another embodiment, the functional domain is a toxin, an epitope, a detectable domain, an enzymatic domain, a cell-stimulating domain or a cell receptor binding domain (e.g., a ligand for a cell receptor).

In another embodiment, the target tissue, organ or cell is diseased, injured, infected, cancerous or mitotically active. In another embodiment, the target tissue, organ or cell is healthy or normal. In another embodiment, the non-target tissue, organ or cell is diseased, injured, infected, cancerous or mitotically active. In another embodiment, the non-target tissue, organ or cell is healthy or normal.

In another aspect, the present invention provides a method of affecting a tissue in a subject comprising administering to said subject a polypeptide comprising a binding domain, a first functional domain and a second functional domain, wherein said binding domain specifically targets said polypeptide to said tissue and said first functional domain exerts an effect on said tissue through a mechanism independent of the mechanism through which said second functional domain exerts an effect on said tissue. In one embodiment, said method is a method of killing said tissue, and said first and said second functional domains are cytotoxic. In another embodiment, said method is a method of activating said tissue. In another embodiment, said method is a method of inducing said tissue. In another embodiment, said method is a method of healing said tissue.

In another aspect, the present invention provides a method of targeting a multifunctional polypeptide to a non-biological surface. In one embodiment, the non-biological surface is metal. In another embodiment, the non-biological surface is plastic. In another embodiment, the non-biological surface is fabric. In another embodiment, the non-biological surface is wood. In another embodiment, the non-biological surface is glass. In another embodiment, the non-biological surface is a mineral. In another embodiment, the non-biological surface is ceramic. In another embodiment, the non-biological surface is soil or dirt. In another embodiment, the non-biological surface is wool, fur or hair. In another embodiment, the non-biological surface is paint, varnish, laquer or sealant. In another embodiment, the non-biological surface is a medical device. In another embodiment, the medical device is implanted in a subject. In another embodiment, the non-biological surface is a diagnostic device.

In another aspect, the present invention provides a method of targeting a multifunctional polypeptide to a plant surface. In one embodiment, the plant surface is a leaf. In another embodiment, the plant surface is a stem. In another embodiment, the plant surface is a root. In another embodiment, the plant surface is a branch. In another embodiment, the plant surface is a flower. In another embodiment, the plant surface is a seed. In another embodiment, the plant surface is a nut. In another embodiment, the plant surface is a vascular surface.

### BRIEF DESCRIPTION OF THE FIGURES

FIG. 1 provides schematic diagrams of two different types of multifunctional polypeptides.

FIG. 2 provides a schematic diagram of a multifunctional polypeptide comprising a number of different active domains.

### DETAILED DESCRIPTION OF THE INVENTION

Unless defined otherwise, all technical and scientific terms used herein have the same meaning as commonly understood by one of ordinary skill in the art to which this invention belongs. All references are incorporated herein by reference in their entireties. Although any methods and materials similar or equivalent to those described herein can be used in the practice or testing of the present invention, the preferred methods and materials are described. For purposes of the present invention, the following terms are defined below.

All single-stranded nucleic acid sequences are written from 5′ to 3′, unless otherwise indicated. The top strand of each double-stranded nucleic acid sequence is written from 5′ to 3′ and the bottom strand from 3′ to 5′, unless otherwise indicated. All peptide sequences are written N-terminus to C-terminus, unless otherwise indicated. Standard one-letter amino acid and nucleic acid abbreviations are used throughout, unless otherwise indicated. In an amino acid sequence, “X” indicates a position that can be occupied by any amino acid residue, including a non-naturally occurring amino acid residue.

A “multifunctional polypeptide” according to the invention is a polypeptide comprising at least one targeted peptide and at least two functional domains from at least two different sources. The two functional domains can be peptides having sequences from different naturally-occurring polypeptides, or one or both of them can be modified sequences from peptides from the same or different naturally-occurring polypeptides, or they can both be artificially created peptide sequences. In certain embodiments the targeted peptide and the functional domains are all from different sources.

A “functional domain” is one or more amino acid residues and/or other components of a multifunctional polypeptide that confer a functionality to the multifunctional polypeptide. The amino acid residues or other components comprising the functional domain need not form a discreet structural domain, i.e., they need not be adjacent to each other in the primary, secondary, tertiary or quaternary structure of the multifunctional polypeptide. Examples of functional domains include binding domains, catalytic domains, antigenic domains, detectable domains and domains that induce an allosteric change in the polypeptide in response to an event or condition (e.g., binding a molecule or a change in pH), or domains that trigger an event at the target, such as an immune response or the activation or suppression of a signal transduction pathway or other cellular response, e.g., apoptosis.

A “binding domain” is one or more amino acid residues and/or other components that allow a multifunctional polypeptide to bind to a microtarget. The amino acid residues or other components comprising the binding domain need not be in proximity to each other in the primary, secondary, tertiary or quaternary structure of the multifunctional polypeptide. A targeted peptide is an example of a binding domain.

A “targeted peptide” is a peptide selected for its ability to bind to a microtarget or target. The targeted peptide can be selected, for example, by screening a library of peptides for sequences that bind to a target. A targeted peptide can comprise peptides from more than one portion of the multifunctional polypeptide that together bind a target or microtarget.

A “microtarget” is a chemical structure or surface that a multifunctional polypeptide can bind to, including, for example, all or part of, or multiple parts of, one or more molecules.

Unless otherwise noted, the term “protein” is used interchangeably here with the terms “peptide” and “polypeptide,” and refers to a molecule comprising two or more amino acid residues joined by a peptide bond.

The term “gene” refers to a DNA sequence that comprises control and coding sequences necessary for the production of a protein, polypeptide or precursor.

The term “oligonucleotide” as used herein is defined as a molecule comprised of two or more deoxyribonucleotides or ribonucleotides. The exact size will depend on many factors, which in turn depends on the ultimate function or use of the oligonucleotide.

Oligonucleotides can be prepared by any suitable method, including, for example, cloning and restriction of appropriate sequences and direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Lett. 22:1859-1862; and the solid support method of U.S. Pat. No. 4,458,066, each incorporated herein by reference. A review of synthesis methods is provided in Goodchild, 1990, Bioconjugate Chemistry 1(3):165-187, incorporated herein by reference.

Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine), nonpolar side chains (e.g., alanine, valine, leucine, isoleucine, proline, phenylalanine, methionine, tryptophan, cysteine, glycine), beta-branched side chains (e.g., threonine, valine, isoleucine) and aromatic side chains (e.g., tyrosine, phenylalanine, tryptophan, histidine). Standard three-letter or one-letter amino acid abbreviations are used herein.

The peptides, polypeptides and proteins of the invention include those comprising one or more non-classical amino acids. Non-classical amino acids include but are not limited to the D-isomers of the common amino acids, α-amino isobutyric acid, 4-aminobutyric acid (4-Abu), 2-aminobutyric acid (2-Abu), 6-amino hexanoic acid (Ahx), 2-amino isobutyric acid (2-Aib), 3-amino propionoic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general.

As used herein, a “point mutation” in an amino acid sequence refers to either a single amino acid substitution, a single amino acid insertion or single amino acid deletion. A point mutation preferably is introduced into an amino acid sequence by a suitable codon change in the encoding DNA. Individual amino acids in a sequence are represented herein as AN, wherein A is the standard one letter symbol for the amino acid in the sequence, and N is the position in the sequence. Mutations within an amino acid sequence are represented herein as A₁ NA₂, wherein A₁ is the standard one letter symbol for the amino acid in the unmutated protein sequence, A₂ is the standard one letter symbol for the amino acid in the mutated protein sequence, and N is the position in the amino acid sequence. For example, a G46D mutation represents a change from glycine to aspartic acid at amino acid position 46. The amino acid positions are numbered based on the full-length sequence of the protein from which the region encompassing the mutation is derived. Representations of nucleotides and point mutations in DNA sequences are analogous.

As used herein, a “chimeric” protein refers to a protein whose amino acid sequence represents a fusion product of subsequences of the amino acid sequences from at least two distinct proteins. A chimeric protein can be produced by, for example, chemical synthesis, direct manipulation of amino acid sequences, or expression from a chimeric gene that encodes the chimeric amino acid sequence.

The term “Ab” or “antibody” refers to polyclonal and monoclonal antibodies, an entire immunoglobulin or antibody or any functional fragment of an immunoglobulin molecule. Examples of such functional entities include complete antibody molecules, antibody fragments, such as Fv, single chain Fv, complementarity determining regions (CDRs), V_{L} (light chain variable region), V_{H} (heavy chain variable region), Fc (see Clynes et al., 2000, Nature Medicine 6:373-74), and any combination of those or any other functional portion of an immunoglobulin peptide.

The term “prodrug” refers to a compound that is converted via one or more steps, for example, enzymatically catalyzed steps, into an active compound that has an increased pharmacological activity relative to the prodrug. A prodrug can comprise a pro-part or inactive moiety and a drug or active drug. Optionally, the prodrug also contains a linker. For example, the prodrug can be cleaved by an enzyme to release an active drug. In a more specific example, prodrug cleavage by the targeted enzyme releases the active drug into the vicinity of the target bound to the targeted enzyme. “Pro-part” and “inactive moiety” refer to the inactive portion of the prodrug after it has been converted. For example, if a prodrug comprises PEG molecule linked by a peptide to an active drug, the pro-part is the PEG moiety with or without a portion of the peptide linker. “Linker” refers to the means connecting the pro-part of a prodrug to the active drug of a prodrug. In one example, the linker is a peptide cleavable by the targeted enzyme, however, it can be any moiety that joins the drug to the propart. In another example, the cleavable segment is a chemical moiety that acts as a substrate for the targeted enzyme, e.g., a β-lactam that is cleaved by β-lactamase. The term “drug” and “active drug” refer to the active moieties of a prodrug. After cleavage by a targeted enzyme, the active drug acts therapeutically upon the targeted tumor, cell, infectious agent or other agent of disease. In another example, the prodrug is chemically modified by the activating enzyme, for example, by oxidation, reduction, phosphorylation, dephosphorylation, the addition of a moiety, or the like. In another example, the prodrug is converted into an intermediate compound by the enzyme. The intermediate compound is converted to the active compound either spontaneously, through contact with other proteins or molecules in the subject (e.g. glucose or metal ions) or conditions (e.g., pH, oxidizing or reducing conditions), through contact with one or more enzymes native to the subject, or through contact with one or more additional activating enzymes or other molecules or substances administered to the subject.

The term “% sequence homology” is used interchangeably herein with the terms “% homology,” “% sequence identity” and “% identity” and refers to the level of amino acid sequence identity between two or more peptide sequences, when aligned using a sequence alignment program. For example, as used herein, 80% homology means the same thing as 80% sequence identity determined by a defined algorithm, and accordingly a homologue of a given sequence has greater than 80% sequence identity over a length of the given sequence. Exemplary levels of sequence identity include, but are not limited to, 60, 70, 80, 85, 90, 95, 98% or more sequence identity to a given sequence.

Exemplary computer programs which can be used to determine identity between two sequences include, but are not limited to, the suite of BLAST programs, e.g., BLASTN, BLASTX, and TBLASTX, BLASTP and TBLASTN, publicly available on the Internet at http://www.ncbi.nlm.nih.gov/BLAST/”. See also Altschul et al., 1990, J. Mol. Biol. 215: 403-10 (with special reference to the published default setting, i.e., parameters w=4, t=17) and Altschul et al., 1997, Nucleic Acids Res., 25:3389-3402. Sequence searches are typically carried out using the BLASTP program when evaluating a given amino acid sequence relative to amino acid sequences in the GenBank Protein Sequences and other public databases. The BLASTX program is preferred for searching nucleic acid sequences that have been translated in all reading frames against amino acid sequences in the GenBank Protein Sequences and other public databases. Both BLASTP and BLASTX are run using default parameters of an open gap penalty of 11.0, and an extended gap penalty of 1.0, and utilize the BLOSUM-62 matrix. See Altschul, et al., 1997.

A preferred alignment of selected sequences in order to determine “% identity” between two or more sequences, is performed using for example, the CLUSTAL-W program in MacVector version 6.5, operated with default parameters, including an open gap penalty of 10.0, an extended gap penalty of 0.1, and a BLOSUM 30 similarity matrix.

The multifunctional polypeptides of the invention are polypeptides that comprise a targeted peptide that binds a target and two or more additional functional domains. Each of the functional domains confers to the polypeptide a function or property, e.g., target binding, a catalytic function, an inducible allosteric change, detectability or toxicity. By combining more than one binding domain and/or more than one other function domain in one polypeptide, the invention provides molecules that have greater specificity and/or activity than previously available.

### Targeted Peptides

The targeted peptides of the invention can comprise any peptide sequence or sequences that allow a multifunctional polypeptide comprising them to bind to one or more particular microtargets when they occur in the context of a target. In one embodiment, the sequence of a targeted peptide is not identical to the sequence of a naturally-occurring target binding peptide, or to the target binding domain of a naturally-occurring target binding protein, and it is not derived from the antigen-binding region of an antibody.

The targeted peptide can be a sequence of amino acids of any length. At the lower end, the length is limited only by the length of sequence needed to bind with sufficient specificity to the microtarget. Depending on the microtarget and the intended use of the multifunctional polypeptide comprising the targeted peptide, theoretically a single amino acid is sufficient, although in most contexts the minimum length is 2, 3, 4, 5, 6, 7, 8, 9, 10 or more amino acids. At the upper end, the length is limited only by the constraints imposed by the multifunctional polypeptide, and can be, e.g., 500, 450,400, 350, 300, 250, 200, 150, 100, 75, 50, 40, 30, 25, 20, 15, 12, 10, 9, 8, 7, 6 or 5 amino acids. Preferably, the targeted peptide is not so large that it unduly interferes with the functional domains of the multifunctional polypeptide.

In one aspect, the present invention provides a targeted peptide comprising a plurality of peptide sequences in the multifunctional polypeptide. That is, the multifunctional polypeptide comprises two or more peptides that, together in the context of the multifunctional polypeptide, bind to a microtarget. In one embodiment, the peptides are close to each other in the primary, secondary, tertiary and/or quaternary structure of the multifunctional polypeptide.

The targeted peptide can bind to its microtarget with any affinity, and the affinity of the targeted peptide can be influenced by the target comprising the microtarget, or by the milieu of the target, e.g., the multifunctional polypeptide can be a milieu-dependent targeted agent as described in U.S. pat. app. Ser. No._______(attorney docket no. 9342-042999) filed concurrently with the present application, incorporated herein by reference in its entirety.

The targeted peptide can bind to its microtarget with a K_{d} of about 100 μM or less, 10 μM or less, 1 μM or less, 100 nM or less, about 90 nM or less, about 80 nM or less, about 70 nM or less, about 60 nM or less, about 50 nM or less, about 40 nM or less, about 30 nM or less, about 20 nM or less, about 10 nM or less, about 5 nM or less, about 1 nM or less or about 0.1 nM or less. In one embodiment, the multifunctional polypeptide comprises a plurality of targeted peptides, each of which binds to its microtarget with a relatively low affinity, such that the multifunctional polypeptide binds to a target comprising the microtargets with a relatively high affinity, as described herein.

A targeted peptide can be selected using any method known in the art. For example, the targeted peptide can be selected from a library of peptides based on its ability to bind a microtarget. The targeted peptide also can be designed using, for example, a “directed evolution” approach, see, e.g., U.S. Pat. Nos. 6,361,974; 6,358,709; 6,352,842; 6,352,842; 6,171,820; 6,156,509; 5,837,500; Brakmann, 2001, Chembiochem. 2:865-71; Farinas et al., 2001, Curr Opin Biotechnol. 12:545-51; Voigt et al., 2001, J Cell Biochem Suppl. 58-63; Cohen et al., 2001, Trends Biotechnol. 19:507-10; Penning et al., 2001, Chem Rev. 101:3027-46, incorporated herein by reference in their entireties, or another iterative approach whereby the microtarget binding properties of a peptide are improved through repeated rounds of mutation and selection.

In one embodiment, the targeted peptide is selected using an affinity maturation approach, e.g., as described in copending U.S. pat. app. Ser. No. (attorney docket no. 9342-040-999), filed concurrently with the present application, incorporated by reference herein in its entirety.

In another embodiment, the targeted peptide is selected using a method provided in copending U.S. pat. app. Ser. No. 10/022,073, filed Dec. 13, 2001, incorporated by reference herein in its entirety.

In another embodiment, the targeted peptide is selected using an affinity maturation method, e.g., a method provided in copending U.S. pat. app. Ser. No. (attorney docket no. 9342-040-999), filed concurrently with the present application, incorporated herein by reference in its entirety.

In another embodiment, the targeted peptide is inserted into an enzyme or polypeptide, e.g., using a loop grafting method, examples of which are provided in copending U.S. pat. app. Ser. No. (attorney docket no. 9342-041-999), filed concurrently with the present application, incorporated herein by reference in its entirety.

### Targets

The targets bound by the multifunctional polypeptide of the present invention can be any substance or composition to which a molecule can be made to bind.

In one aspect, the target is a surface. In one embodiment, the surface is a biological surface. In another embodiment, the biological surface is a surface of an organ. In another embodiment, the biological surface is a surface of a tissue. In another embodiment, the biological surface is a surface of a cell. In another embodiment, the biological surface is a surface of a diseased organ, tissue or cell. In another embodiment, the biological surface is a surface of a normal or healthy organ, tissue or cell. In another embodiment, the surface is a macromolecule in the interstitial space of a tissue. In another embodiment, the biological surface is the surface of a virus or pathogen. In another embodiment, the surface is a non-biological surface. In another embodiment, the non-biological surface is a surface of a medical device. In another embodiment, the medical device is a therapeutic device. In another embodiment, the therapeutic device is an implanted therapeutic device. In another embodiment, the medical device is a diagnostic device. In another embodiment, the diagnostic device is a well or tray.

Sources of cells or tissues include human, animal, bacterial, fungal, viral and plant. Tissues are complex targets and refer to a single cell type, a collection of cell types or an aggregate of cells generally of a particular kind. Tissue may be intact or modified. General classes of tissue in humans include but are not limited to epithelial tissue, connective tissue, nerve tissue, and muscle tissue.

In another aspect, the target is a cancer-related target. The cancer-related target can be any target that a composition of the invention binds to as part of the diagnosis, detection or treatment of a cancer or cancer-associated condition in a subject, for example, a cancerous cell, tissue or organ, a molecule associated with a cancerous cell, tissue or organ, or a molecule, cell, tissue or organ that is associated with a cancerous cell, tissue or organ (e.g., a tumor-bound diagnostic or therapeutic molecule administered to a subject or to a biopsy taken from a subject, or a healthy tissue, such as vasculature, that is associated with cancerous tissue). Examples of cancer-related targets are provided in U.S. Pat. No. 6,261,535, which is incorporated herein by reference in its entirety.

The cancer-related target can be related to any cancer or cancer-associated condition. Examples of types of cancers include carcinomas, sarcomas, myelomas, leukemias, lymphomas and mixed type cancers.

In one embodiment, the cancer is a bone cancer, for example, Ewing's sarcoma, osteosarcoma and rhabdomyosarcoma and other soft-tissue sarcomas. In another embodiment, the cancer is a brain tumor, for example, oligodendroglioma, ependymoma, menengioma, lymphoma, schwannoma or medulloblastoma. In another embodiment, the cancer is breast cancer, for example, ductal carcinoma in situ of the breast. In another embodiment, the cancer is an endocrine system cancer, for example, adrenal, pancreatic, parathyroid, pituitary and thyroid cancers. In another embodiment, the cancer is a gastrointestinal cancer, for example, anal, colorectal, esophogeal, gallbladder, gastric, liver, pancreatic, and small intestine cancers. In another embodiment, the cancer is a gynecological cancer, for example, cervical, endometrial, uterine, fallopian tube, gestational trophoblastic disease, choriocarcinoma, ovarian, vaginal, and vulvar cancers. In another embodiment, the cancer is a head and neck cancer, for example, laryngeal, oropharyngeal, parathryroid or thyroid cancer. In another embodiment, the cancer is a leukemic cancer, for example, acute lymphocytic leukemia, acute myelogenous leukemia, chronic lymphocytic leukemia, chronic myelogenous leukemia, hairy cell leukemia, or a myeloproliferative disorder. In another embodiment, the cancer is a lung cancer, for example, a mesothelioma, non-small cell small cell lung cancer. In another embodiment, the cancer is a lymphoma, for example, AIDS-related related lymphoma, cutaneous T cell lymphoma/mucosis fungoides, Hodgkin's disease, or non-Hodgkin's disease. In another embodiment, the cancer is metastatic cancer. In another embodiment, the cancer is a myeloma, for example, a multiple myeloma. In another embodiment, the cancer is a pediatric cancer, for example, a brain tumor, Ewing's sarcoma, leukemia (e.g., acute lymphocytic leukemia or acute myelogenous leukemia), liver cancer, a lymphoma (e.g., Hodgkin's lymphoma or non-Hodgkin's lymphoma), neuroblastoma, retinoblastoma, a sarcoma (e.g., osteosarcoma or rhabdomyosarcoma), or Wilms' Tumor. In another embodiment, the cancer is penile cancer. In another embodiment, the cancer is prostate cancer. In another embodiment, the cancer is a sarcoma, for example, Ewing's sarcoma, osteosarcoma, rhabdomyosarcoma and other soft-tissue sarcomas. In another embodiment, the cancer is a skin cancer, for example, cutaneous T cell lymphoma, mycosis fungoides, Kaposi's sarcoma or melanoma. In another embodiment, the cancer is testicular cancer. hi another embodiment, the cancer is thyroid cancer, for example, papillary, follicular, medullary, or anaplastic or undifferentiated thyroid carcinoma. In another embodiment, the cancer is urinary tract cancers, for example, bladder, kidney or urethral cancers. In another embodiment, the cancer or cancer-related condition is ataxiatelangiectasia, carcinoma of unknown primary origin, Li-Fraumeni syndrome, or thymoma.

In another aspect, the cancer-related target is a molecule associated with a cancerous cell or tissue. In one embodiment, the molecule is a tumor or tumor stroma antigen, for example, GD2, Lewis-Y, 72 kd glycoprotein (gp 72, decay-accelerating factor, CD55, DAF, C3/C5 convertases), CO17-1A (EpCAM, 17-1A, EGP-40), TAG-72, CSAg-P (CSAp), 45 kd glycoprotein, HT-29 ag, NG2, A33 (43 kd gp), 38 kd gp, MUC-1, CEA, EGFR (HER 1), HER2, HER3, HER4, HN-1 ligand, CA125, syndecan-1, Lewis X, PgP, FAP stromal Ag (fibroblast activation protein), EDG receptors (endoglin receptors), ED-B, laminin-5 (gamma2), cox-2 (+LN-5), PgP (P-glycoprotein), alphaVbeta3 integrin, alphaVbeta5, integrin, uPAR (urokinase plasminogen activator receptor), endoglin (CD 105), folate receptor osteopontin (EDG 1,3), p97 (melanotransferrin), farnesyl transferase or a molecule in an apoptotic pathway (e.g., a death receptor, fas, caspase or bcl-2) or a lectin.

In another aspect, the target is a hematopoietic cell. Hematopoietic cells encompass hematopoietic stem cells (HSCs), erythrocytes, neutrophils, monocytes, platelets, mast cells, eosinophils, basophils, B and T cells, macrophages, and natural killer cells. In one embodiment, the HSC has a surface antigen expression profile of CD34⁺ Thy-1⁺, and preferably CD34⁺ Thy-1⁺ Lin⁻. Lin⁻ refers to a cell population selected on the basis of the lack of expression of at least one lineage specific marker. Methods for isolating and selecting HSCs are well known in the art and reference is made to U.S. Pat. Nos. 5,061,620, 5,677,136, and 5,750,397, each of which is incorporated herein in its entirety.

In another aspect, the target is a molecule. In one embodiment, the molecule is an organic molecule. In another embodiment, the molecule is a biological molecule. In another embodiment, the biological molecule is a cell-associated molecule. In another embodiment, the cell-associated molecule is associated with the outer surface of a cell. In another embodiment, the cell-associated molecule is part of the extracellular matrix. In another embodiment, the cell-associated molecule is associated with the outer surface of a cell is a protein. In another embodiment, the protein is a receptor. In another embodiment, the cell-associated molecule is specific to a type of cell in a subject. In another embodiment, the type of cell is a diseased cell. In another embodiment, the diseased cell is a cancer cell. In another embodiment, the diseased cell is an infected cell. Other molecules that can serve as targets according to the invention include, but are not limited to, proteins, peptides, nucleic acids, carbohydrates, lipids, polysaccharides, glycoproteins, hormones, receptors, antigens, antibodies, toxic substances, metabolites, inhibitors, drugs, dyes, nutrients and growth factors.

Non-limiting examples of protein and chemical targets encompassed by the invention include chemokines and cytokines and their receptors. Cytokines as used herein refer to any one of the numerous factors that exert a variety of effects on cells, for example inducing growth or proliferation. Non-limiting examples include interleukins (IL), IL-2, IL-3, IL-4 IL6, IL-10, IL-12, IL-13, IL-14 and IL-16; soluble IL-2 receptor; soluble IL-6 receptor; erythropoietin (EPO); thrombopoietin (TPO); granulocyte macrophage colony stimulating factor (GM-CSF); stem cell factor (SCF); leukemia inhibitory factor (LIF); interferons; oncostatin M (OM); the immunoglobulin superfamily; tumor necrosis factor (TNF) family, particularly TNF-α; TGFβ; and IL-1α; and vascular endothelial growth factor (VEGF) family, particularly VEGF (also referred to in the art as VEGF-A), VEGF-B, VEGF-C, VEGF-D and placental growth factor (PLGF). Cytokines are commercially available from several vendors including Amgen (Thousand Oaks, Calif.), immunex (Seattle, Wash.) and Genentech (South San Francisco, Calif.). Particularly preferred are VEGF and TNF-α. Antibodies against TNF-α show that blocking interaction of the TNF-α with its receptor is useful in modulating over-expression of TNF-α in several disease states such as septic shock, rheumatoid arthritis, or other inflammatory processes. VEGF is an angiogenic inducer, a mediator of vascular permeability, and an endothelial cell specific mitogen. VEGF has also been implicated in tumors. Targeting members of the VEGF family and their receptors may have significant therapeutic applications, for example blocking VEGF may have therapeutic value in ovarian hyper stimulation syndrome (OHSS). Reference is made to N. Ferrara et al., (1999) Nat. Med. 5:1359 and Gerber et al., (1999) Nat. Med. 5:623. Other preferred targets include cell-surface receptors, such as T-cell receptors.

Chemokines are a family of small proteins that play an important role in cell trafficking and inflammation. Members of the chemokine family include, but are not limited to, IL-8, stomal-derived factor-1(SDF-1), platelet factor 4, neutrophil activating protein-2(NAP-2) and monocyte chemo attractant protein-1 (MCP-1).

Other protein and chemical targets include, but are not limited to: immunoregulation modulating proteins, such as soluble human leukocyte antigen (HLA, class I and/or class II, and non-classical class I HLA (E, F and G)); surface proteins, such as soluble T or B cell surface proteins; human serum albumin; arachadonic acid metabolites, such as prostaglandins, leukotrienes, thromboxane and prostacyclin; IgE, auto or alloantibodies for autoimmunity or allo- or xenoimmunity, Ig Fc receptors or Fc receptor binding factors; G-protein coupled receptors; cell-surface carbohydrates; angiogenesis factors; adhesion molecules; ions, such as calcium, potassium, magnesium, aluminum, and iron; fibril proteins, such as prions and tubulin; enzymes, such as proteases, aminopeptidases, kinases, phosphatases, DNAses, RNAases, lipases, esterases, dehydrogenases, oxidases, hydrolases, sulphatases, cyclases, transferases, transaminases, carboxylases, decarboxylases, superoxide dismutase, and their natural substrates or analogs; hormones and their corresponding receptors, such as follicle stimulating hormone (FSH), leutinizing hormone (LH), thyroxine (T4 and T3), apolipoproteins, low density lipoprotein (LDL), very low density lipoprotein (VLDL), cortisol, aldosterone, estriol, estradiol, progesterone, testosterone, dehydroepiandrosterone (DHBA) and its sulfate (DHEA-S); peptide hormones, such as renin, insulin, calcitonin, parathyroid hormone (PTH), human growth hormone (hGH), vasopressin and antidiuretic hormone (AD), prolactin, adrenocorticotropic hormone (ACTH), LHRH, thyrotropin-releasing hormone (THRH), vasoactive intestinal peptide (VIP), bradykinin and corresponding prohormones; catechcolamines such as adrenaline and metabolites; cofactors including atrionatriutic factor (AdF), vitamins A, B, C, D, E and K, and serotonin; coagulation factors, such as prothrombin, thrombin, fibrin, fibrinogen, Factor VIII, Factor IX, Factor XI, and von Willebrand factor; plasminogen factors, such as plasmin, complement activation factors, LDL and ligands thereof, and uric acid; compounds regulating coagulation, such as hirudin, hirulog, hementin, hepurin, and tissue plasminigen activator (TPA); nucleic acids for gene therapy; compounds which are enzyme antagonists; and compounds binding ligands, such as inflammation factors; and receptors and other proteins that bind to one or more of the preceding molecules.

Non-human derived targets include without limitation drugs, especially drugs subject to abuse, such as cannabis, heroin and other opiates, phencyclidine (PCP), barbiturates, cocaine and its derivatives, and benzadiazepine; toxins, such as heavy metals like mercury and lead, arsenic, and radioactive compounds; chemotherapeutic agents, such as paracetamol, digoxin, and free radicals; bacterial toxins, such as lipopolysaccharides (LPS) and other gram negative toxins, Staphylococcus toxins, Toxin A, Tetanus toxins, Diphtheria toxin and Pertussis toxins; plant and marine toxins; snake and other venoms, virulence factors, such as aerobactins, or pathogenic microbes; infectious viruses, such as hepatitis, cytomegalovirus (CMV), herpes simplex virus (HSV types 1, 2 and 6), Epstein-Barr virus (EBV), varicella 2 zoster virus (VZV), human immunodeficiency virus (HIV-1, -2) and other retroviruses, adenovirus, rotavirus, influenzae, rhinovirus, parvovirus, rubella, measles, polio, pararyxovirus, papovavirus, poxvirus and picomavirus, prions, plasmodia tissue factor, protozoans, such as Entamoeba histolitica, Filaria, Giardia, Kalaazar, and toxoplasma; bacteria, gram-negative bacteria responsible for sepsis and nosocomial infections such as E. coli, Acynetobacter, Pseudomonas, Proteus and Klebsiella, also gram-positive bacteria such as Staphylococcus, Streptococcus, Meningococcus and Llycobacteria, Chlamydiae Legionnella and Anaerobes; fungi such as Candida, Pneumocystis, Aspergillus, and Mycoplasma.

In one aspect the target includes an enzyme such as proteases, aminopeptidases, kinases, phosphatases, DNAses, RNAases, lipases, esterases, dehydrogenases, oxidases, hydrolases, sulphatases, cellulases, cyclases, transferases, transaminases, carboxylases, decarboxylases, superoxide dismutase, and their natural substrates or analogs. Particularly preferred enzymes include hydrolases, particularly alpha/beta hydrolases; serine proteases, such as subtilisins, and chymotrypsin serine proteases; cellulases; and lipases.

In another embodiment, the target is a non-biological material. In another embodiment, the non-biological material is a fabric. In another embodiment, the fabric is a natural fabric. In another embodiment, the fabric is cotton. In another embodiment, the fabric is silk. In another embodiment, the fabric is wool. In another embodiment, the fabric is a non-natural fabric. In another embodiment, the fabric is nylon. In another embodiment, the fabric is rayon. In another embodiment, the fabric is polyester. In another embodiment, the non-biological material is a plastic. In another embodiment, the non-biological material is a ceramic. In another embodiment, the non-biological material is a metal. In another embodiment, the non-biological material is rubber.

In another embodiment the target is a microcircuit. This circuit can be in its finished form or in any stage of circuit manufacturing. The multifunctional polypeptide can be used to remove or deposit a compound onto the circuit, for example, an n-type dopant (e.g., arsenic, phosphorus, antimony, titanium or other donor atom species) or a p-type dopant (e.g., boron, aluminum, gallium, indium or other acceptor atom species). See, e.g., van Zant, 2000, Microchip Fabrication, McGraw-Hill, New York, incorporated herein by reference in its entirety.

In another embodiment, the target is not an antibody (e.g., a polyclonal antibody, a monoclonal antibody, an scFv, or another antigen-binding fragment of an antibody).

### Microtargets

The microtarget is the portion or portions of the target that is bound by the binding domain. The microtarget can comprise any kind of molecule, or a portion of a molecule, or a plurality of molecules or portions of molecules, for example, all or part of any of the targets discussed above. The microtarget can be known or unknown to the operator. Examples of types of microtargets include peptides, polypeptides or proteins (e.g., antibodies, antibody fragments (for example, single chain antibody variable region fragment (scFv), ligand-binding peptides, polypeptides or proteins, receptor-binding peptides, polypeptides or proteins or an epitope), organic molecules (e.g., sugars, lipids, amino acids, nucleotides or small organic molecules) or inorganic molecules. In one embodiment, the microtarget is associated with a cell, for example, a cell surface marker. In a more particularly defined embodiment, the microtarget associated with a cell is a tumor antigen (e.g., a carcinoembryonic antigen, p97, A33, or MUC-1).

### Functional Domains

The functional domains of the present invention comprise one or more amino acid residues and/or other components that together confer a functionality to the multifunctional polypeptide, regardless of their locations in the multifunctional polypeptide. Thus, there is no requirement that the amino acids, or other components, of a functional domain be adjacent to each other in the primary, secondary, tertirary or quaternary structure of the multifunctional polypeptide. Examples of functional domains include binding domains (e.g., a targeted peptide of the present invention), catalytic domains, molecular modification recognition domains, cleavage recognition domains, reporter domains, immunomodulatory domains and sensing domains. Functional domains of the present invention do not, generally, include peptide sequences that merely, for example, allow the multifunctional protein to adopt a particular secondary or tertiary conformation or are glycosylation, phosphorylation or cleavage sites, unless these functions are reponsive to environmental stimuli, as discussed below. The sequence of a functional domain of the present invention can be identical to the sequence of a peptide comprised by a naturally-occurring protein, a modification of the sequence of a peptide comprised by a naturally-occurring protein (e.g., a binding or catalytic domain that has been mutated or otherwise changed to improve or alter its activity or specificity, or to improve its activity in the context of the isolated peptide or the multifunctional polypeptide) or it can be an artificial sequence.

In one aspect, the present invention provides a functional domain that is a binding domain. In one embodiment, the functional domain is a targeted peptide of the present invention as described herein. In another embodiment, the multifunctional polypeptide comprises two or more binding domains (e.g., two targeted polypeptides). In another embodiment, each of the binding domains binds a different microtarget, and each of the binding domains binds its microtarget with a relatively low affinity. In another embodiment, the two or more microtargets bound by the two or more binding domains occur together in a target cell, tissue or organ type in a subject, but a non-target cell, tissue or organ type in the subject displays fewer than all, or none, of the target molecules, such that the multifunctional polypeptide binds to the target cell, tissue or organ with a higher affinity or higher specificity than it binds to the non-target cell, tissue or organ.

In another aspect, the present invention provides a functional domain that has a catalytic activity. The catalytic activity can be any catalytic activity known in the art, e.g., a protease activity, a kinase activity, a phosphatase activity, a hydrolase activity, a metabolic activity, an oxidase activity, a reductase activity, a glycolyase activity or a peroxidase activity.

In another aspect, the present invention provides a functional domain that is a molecular modification recognition domain. Examples of molecular modification recognition domains include domains that recognize a peptide, polypeptide or protein that has been post-translationally modified (or, conversely, that recognize a peptide, polypeptide or protein that has not been post-translationally modified), e.g., a peptide, polypeptide or protein that has been phosphorylated, dephosphorylated, glycosylated, deglycosylated, cleaved (e.g., by a protease), ligated, acylated, deacylated, or differentially spliced or processed.

In another aspect, the present invention provides a functional domain that is a cleavage recognition domain, i.e., a sequence that is recognized and cleaved by a protease. In one example, the multifunctional polypeptide comprises a first functional domain that is a cleavage recognition domain and a second functional domain whose activity is affected when the cleavage recognition domain is cleaved, for example, the second functional domain is activated when the cleavage recognition domain is cleaved. In one embodiment, the protease that recognizes the cleavage recognition domain is associated with the target, such that cleavage of the cleavage recognition domain occurs preferentially at or near the target.

In another aspect, the present invention provides a functional domain that comprises a an inhibitor of an enzyme, for example, a competitive inhibitor or a non-competitive inhibitor. In one embodiment, the functional domain is a suicide inhibitor, i.e., a molecule that serves as a substrate for the enzyme, but which inactivates the enzyme when it is modified by the enzyme. Examples of suicide inhibitors include, e.g., phenylmethyl sulfonyl fluoride (PMSF), a protease inhibitor. In another embodiment, the inhibitor portion of the multifunctional enzyme also serves to increase the activity of the multifunctional polypeptide against the target, e.g., by binding to the inhibited enzyme, thus targeting the multifunctional polypeptide to the target, or by activating a function of the multifunctional polypeptide upon binding or inhibiting the enzyme.

In another aspect, the present invention provides a functional domain that activates or inhibits a pathway or function of the target. In one embodiment, the target is a cell, tissue or organ. Examples of pathways or functions that can be activated or inhibited include DNA replication, transcription, RNA processing, translation, protein modification, intracellular traficking, receptor recycling, signal transduction, morphogenesis, mitosis, meiosis, migration, haptotaxis, secretion, endocytosis, differentiation, determination, apoptosis, proliferation, metastasis. Examples of functional domains that can activate or inhibit a function or pathway of a target cell, tissue or organ include chemokines, cytokines, growth factors, hormones, mitogens, motogens, activins, inhibins, morphogens, transcription factors, metal ions, receptor agonists, receptor antagonists, protease inhibitors, steroid hormone receptor binding ligands, peptidomimetics, apoptotic factors and allosteric binders. A functional domain that is an activating or inhibiting domain also can serve as a target binding domain.

In another embodiment, the multifunctional polypeptide comprises two or more identical functional domains such that the avidity of the multifunctional polypeptide for the inhbited molecule is increased. In another embodiment, the multifunctional polypeptide comprises two or more non-identical functional domains, each inhibiting a different target-associated function or pathway, such that the selectivity and/or functionality of the multifunctional polypeptide for the target is increased.

In another aspect, the present invention provides a functional domain that is a reporter domain. The reporter domain can have any activity or property that allows it to be detected. Examples of reporter domains include epitopes, haptens, radioactive groups, fluorescent molecules (e.g., green fluorescent protein or a derivative thereof), light-emitting molecules, molecules detectable by a spectroscopic technique (e.g., infrared, nuclear magnetic resonance or mass spectroscopy) or a molecule that specifically binds another molecule (e.g., a histidine tag, which binds a nickel column, or a biotin moiety, which binds to strepavidin or avidin).

In another aspect, the present invention provides a functional domain that activates a function or pathway that acts on the target, for example, a functional domain that, when bound to a target cell, tissue or organ, activates an immune response against it (e.g., an immunogenic epitope, a tumor marker or a molecule associated with immune system regulation, such as a molecule that upregulates a localized immune response) or an immuno-protective response. In one embodiment, the functional domain is an allogenic or allotypic Class I Major Histocompatibility Complex (MHC) molecule, such that a target cell, tissue or organ bound by the multifunctional polypeptide is recognized as foreign by the immune system. In another embodiment, the functional domain is an immunomodulatory domain. Examples of immunomodulatory domains include domains that are cleavable by a protease, are labile to environmental conditions (e.g., reducing conditions, oxidizing conditions or pH).

In another aspect, the present invention provides a functional domain that alters the environment of a target, for example, the pH, temperature or concentration of ions or metabolites.

In another aspect, the present invention provides a functional domain that allows a multifunctional polypeptide to respond to an environmental stimulus. Examples of these types of functional domains include domains that allow the multifunctional polypeptide to adopt a secondary or tertiary conformation, be phosphorylated or dephosphorylated, be glycosylated or deglycosylated, or be cleaved or conjugated, in response to an environmental stimulus. Examples of environmental stimuli include, for example, an activity that is present at or near the target, but not at or near a non-target (or, conversely, an activity that is absent at or near a target, but is present at or near a non-target), e.g., a kinase, phosphatase, glycosylase, deglycosylase, protease or ligase activity. Alternatively, the functional domain can allow one or more of these activities to act on the multifunctional polypeptide, or allow it to change its secondary or tertiary conformation, in response to binding a molecule. The functional domain that allows this effect to happen may or may not be the same domain that binds to the molecule. In one embodiment, the functional domain comprises a peptide that is specifically cleaved by a protease activity associated with a target cell, tissue or organ type, such that when the multifunctional polypeptide is cleaved by the protease, another functional domain is activated (e.g., a binding domain). In another embodiment, the functional domain comprises a peptide that is cleaved by a protease that is not associated with a target cell, tissue or organ, such that when the multifunctional polypeptide is cleaved by the protease, a functional domain of the multifunctional polypeptide is inactivated.

In another aspect, the present invention provides a functional domain that allows the multifunctional polypeptide to bind to a protein, e.g., to form heterodimers, heterotrimers, heterotetramers, homodimers, homodimers, homotrimers or homotetramers. In one embodiment, the functional domain allows the multifunctional polypeptide to oligomerize in response to a stimulus, e.g., binding to the target, entering the target, entering the milieu of the target, or being modified by an activity associated with the target. In another embodiment, the oligomerization of the multifunctional polypeptide is cooperative.

In another aspect, the present invention provides a functional group that has a cell-killing activity, either on its own or in combination with another treatment, for example, ricin, chemo- or radiation sensitizers or re-sensitizers, or perforin, doxorubicin, taxol, vincristine, vinblastine, other metabolic inhibitors. In one embodiment, the multifunctional polypeptide of the invention is administered to a subject who has ovarian cancer and has failed platinum therapy due to overexpression of glutathione-S-transferase, wherein the multifunctional polypeptide down regulates or inhibits glutathione-S-transferase. In a more particularly defined embodiment, the multifunctional polypeptide comprises an antimalarial molecule.

In another aspect, the present invention provides a functional group that kills or inactivates cells that take up the multifunctional polypeptide. For example, the multifunctional polypeptide can kill or inactivate an antigen-presenting cell and thus interfere with the development of an immune response.

In another aspect, the present invention provides a functional group that comprises a non-proteinaceous molecule, for example, a molecule that improves a property or function or that imparts a property or function to the multifunctional polypeptide, e.g., increased or decreased stability or lability, binding to a target, catalytic activity, toxicity, or detectability. In one embodiment, the non-proteinaceous functional group is radioactive or fluorescent. These molecules can be attached to the multifunctional polypeptide by any means. In one embodiment, the molecule is attached via a covalent bond, either directly or through a linking group.

In another aspect, the present invention provides a functional domain that is photoreactive, for example, a functional domain that is activated or inactivated in response to light. In one embodiment, the functional group is photolabile, i.e., it is hydrolyzed or otherwise cleaved in response to exposure to light. The light can be, for example, visible light, ultraviolet light, infrared light, ambient light, or light from a special light source. Activation of the photoreactive domain can have any desired effect on the multifunctional polypeptide, for example, it can activate another functional domain of the multifunctional polypeptide, inactivate another functional domain of the multifunctional polypeptide or allow the multifunctional polypeptide to be detected.

In another aspect, the present invention provides a multifunctional polypeptide that can polymerize. In one embodiment, the multifunctional polypeptide comprises a functional domain that allows dimerization. In another embodiment, the multifunctional polypeptide comprises functional domains that allow larger polymers to be formed. For example, the multifunctional polypeptide can comprise two domains, each capable of binding the other when present on different multifunctional polypeptide molecules. In another embodiment, polymerization of the multifunctional polypeptide is affected by another function of the multifunctional polypeptide, for example, binding to a target or to another multifunctional polypeptide. In another embodiment, binding of a multifunctional polypeptide to a target, or to another multifunctional polypeptide, is cooperative. In another embodiment, polymerization of the multifunctional polypeptide can form coating on a target surface, for example, a protective coating (e.g., resistance to infection, parasites, corrosion, oxidation, heat or cold), an insulating coating or a conducting coating.

### Methods of Using Multifunctional Polypeptides

The multifunctional polypeptides of the present invention can be used for any purpose. Examples of useful applications of the multifunctional polypeptides of the invention include therapeutic or diagnostic applications, the fabrication of therapeutic or diagnostic molecules, or other small molecules, the fabrication of computer chips or other “nanotechnology”—related applications, industrial applications (e.g., coating ship hulls to protect them from damage from water, salt water, oxidation, light or barnacles and other marine organisms), creating polymers (e.g., for molecular bandages, molecular sutures, functional cosmetics, cosmeceuticals or skin products), treating wood products or making or processing agricultural chemicals (e.g., pesticides and herbicides).

In one aspect, the present invention provides multifunctional polypeptides useful for therapeutic applications. The multifunctional polypeptides of the present invention are well-suited to a wide variety of therapeutic applications because they provide a novel combination of functionalities in a single molecule.

Multifunctional polypeptides of the present invention can be designed to treat any disease, infection or injury, including, for example, cancer, cardiovascular disease, diseases of the central nervous system, gastrointestinal disorders, infectious diseases and immunological disorders including asthma or inflammation.

In one embodiment, the multifunctional polypeptide is useful for treating cancer. The multifunctional polypeptide can have any activity or combination of activities that has a desired effect on a cancerous target, e.g., a cancerous cell, tissue, organ or tumor. For example, the multifunctional polypeptide can kill the cancerous target or inhibit its growth, spread or metastasis. The multifunctional polypeptide can, for example, bind to a cancerous target-associated marker, e.g., a tumor-associated stromal marker such as α-fetoprotein, the Ed-B oncofetal domain of fibronectin or a Ed-B oncofetal domain antibody binding fragment, see Marty et al., 2001, Protein Expression and Purification 21:156-64, urokinase-type plasminogen activator, fetal antigens, gastrin-releasing peptide receptors, vascular endothelial growth factor receptors, integrins, extracellular matrix epitopes and the like, particularly those that are upregulated as a component of tumor resistance and/or response to a prior therapy, or that are localized to the invasive hotspot regions of tumors, or that are markers associated with quiescent tumors, as these areas may be able to better escape therapy due to poorer biodistribution or lower metabolic rate.

The multifunctional polypeptides of the invention can be designed to exert any desired effect on a target cell, tissue or organ. For example, the multifunctional polypeptide can be designed to kill the target cell, tissue or organ, or to inhibit or activate a pathway or function of the target cell, tissue or organ.

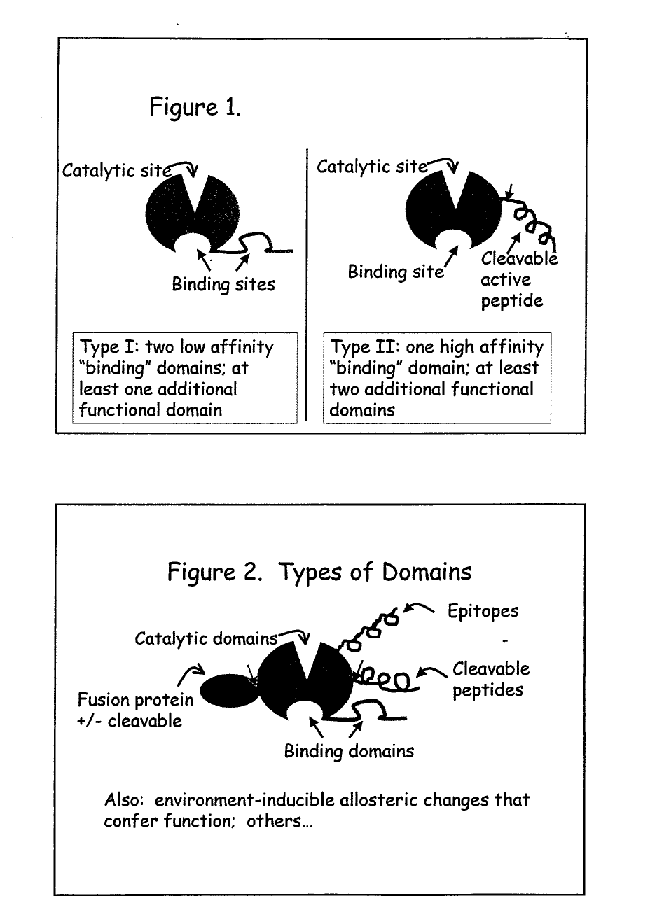

In one embodiment, the multifunctional polypeptide comprises a targeted peptide and two or more functional domains, wherein each functional domain, independently of the others, has an activity that produces a desired effect, and, in the context of the multifunctional polypeptide, the two or more functional domains together produce an effect greater in magnitude than would be produced by a molecule comprising any of the functional domains alone. See FIG. 1. For example, a multifunctional polypeptide useful for killing a target cell could comprise, in addition to one or more targeted peptides that bind to the target cell, a catalytic function that activates a prodrug, a toxic peptide (e.g., ricin), an epitope that elicits an immune response or a signaling domain that triggers apoptosis. While each of these activities, on its own, is useful for killing the target cell, the combination of more than one of them, or all of them, in one molecule provides a much more effective means for killing the target cell.

In another embodiment, the multifunctional polypeptide comprises two or more binding domains, each binding a different target molecule, and each of the binding domains binds its target molecule with a relatively low affinity, wherein the two or more target molecules bound by the two or more binding domains occur together in a target cell, tissue or organ type in a subject, but a non-target cell, tissue or organ type in the subject displays fewer than all, or none, of the target molecules, such that the multifunctional polypeptide binds to the target cell, tissue or organ with a higher affinity and specificity than it binds to the non-target cell, tissue or organ. See FIG. 1.

In another aspect, the invention provides a multifunctional polypeptide that acts in more than one “stage.” For example, the multifunctional polypeptide can act through a first stage that affects the target in one way, then act through a second stage that affects the target in a second way, such that the combined effect of the multifunctional polypeptide's actions at the first and second stages is a greater effect on the target than could be achieved through either stage alone. In one embodiment, the multifunctional polypeptide binds to the outside of a target cell, or a component of its extracellular matrix or extracellular milieu, where a first functional domain is active (e.g., the first functional domain inhibits a receptor or triggers an intracellular signaling pathway), then the multifunctional polypeptide is internalized by the cell, where a second functional domain is active (e.g., the second functional domain inhibits and enzyme or is a cytotoxic peptide).

In another aspect, the multifunctional polypeptides of the invention are used to affect the toxicity of a chemotherapeutic molecule. In one embodiment, the multifunctional polypeptide binds to and protects healthy or normal cells, tissues or organs from the effects of the chemotherapeutic molecule. For example, the multifunctional polypeptide can comprise a functional domain that protects the target tissue from the toxic effects of the toxin by inactivating the toxin (e.g., by binding it or by chemically modifying it to a less toxic form) or by altering the target so that it is less susceptible to the toxic effects of the toxin (e.g., by blocking a receptor or other component of the target through which the toxin exerts its toxic effects). In another embodiment, the multifunctional polypeptide increases the toxicity of a chemotherapeutic molecule. For example, the multifunctional polypeptide can target a cell, tissue or organ that detoxifies a chemotherapeutic molecule, thereby reducing the ability of the target to detoxify the chemotherapeutic. In another example, the multifunctional polypeptide targets the cell, tissue or organ that is the target of the chemotherapeutic such that efflux of the chemotherapeutic is reduced.

In another embodiment, the multifunctional polypeptides of the invention are targeted enzymes, that is, they are enzymes that have been modified such that they bind to a target with a higher affinity than a pre-modified enzyme binds to the target, but retain their enzymatic activity. Examples of targeted enzymes and methods for making them are provided in copending U.S. patent application Ser. No. 10/022,073, filed Dec. 13, 2001, incorporated herein by reference in its entirety.

In another embodiment, the multifunctional polypeptides of the invention comprise a functional domain that activates a prodrug, that is, a drug that is administered in a less active form and is converted into a more active form by the action of a multifunctional polypeptide. In one embodiment, the multifunctional polypeptide of the invention is a targeted enzyme prodrug therapy (TEPT) molecule. Examples of prodrugs and TEPT molecules, and methods of making TEPT molecules, are provided in copending U.S. patent application Ser. No. 10/022,097, filed Dec. 13, 2001, incorporated herein by reference in its entirety.

In another embodiment, the multifunctional polypeptides of the invention comprise a milieu-dependent domain, that is, a domain that is more active in a first milieu than in a second milieu. Examples of differences between the first milieu and the second milieu include, e.g., pH, concentration of an ion, solute, metabolite, or other molecule and temperature. In another embodiment, the difference is a difference in pH. In another embodiment, the milieu-dependent domain is more active in the relatively more acidic milieu of a cancer cell than in the relatively less acidic milieu of a non-cancer cell. In another embodiment, the milieu-dependent domain is a functional domain, e.g., a catalytic domain. In another embodiment, the milieu-dependent domain is a binding domain, e.g., a targeted peptide. Examples of milieu-dependent binding domains include those provided in copending U.S. pat. app. Ser. No. _______(attorney docket no. 9342-042-999), filed concurrently with the present application, incorporated by reference herein in its entirety.

In another embodiment, the multifuncational polypeptides of the invention are used to detect and/or identify novel targets or microtargets, for example, surface features that are unique to a surface. For example, a “tag” library (e.g.., a library of peptides) can be attached to a targeted peptide that binds to a target with a relatively low affinity, forming a tagged targeted library. The target can then be contacted with the tagged targeted library, and members of the library that bind to the target with a greater affinity than the targeted peptide without a tag binds the target can be identified.

We claim:

1) A method of making a multifunctional polypeptide comprising
a) selecting from a library of peptides a peptide that binds to a target, and
b) making a multifunctional polypeptide that comprises the sequence of said peptide selected in step (a) and at least two functional domains.

2) The method of claim 1 wherein the target comprises a molecule or surface associated with a tissue, an organ or a cell.

3) The method of claim 2 wherein the tissue, organ or cell is diseased, injured, infected, cancerous, healthy or normal.

4) A method of selectively targeting a target comprising contacting said target with a multifunctional polypeptide wherein said multifunctional polypeptide comprises a first low affinity targeted peptide for a first microtarget and a second low affinity targeted peptide for a second microtarget, said target comprises said first and said second microtargets, and a non-target does not comprise both said first and said second microtargets, such that the affinity and specificity of said multifunctional polypeptide is higher for said target than it is for said non-target.

5) The method of claim 4 wherein the multifunctional polypeptide is administered to a subject and said subject comprises said target and said non-target.

6) The method of claim 5 wherein the target is a diseased, injured or infected tissue, organ or cell.

7) The method of claim 6 wherein the target is a cancerous tissue, organ cell or tumor.

8) The method of claim 7 wherein the first microtarget is p97.

9) The method of claim 5 wherein the non-target is a normal or healthy tissue, organ or cell.

11) The method of claim 5 wherein the multifunctional polypeptide further comprises a functional domain.

12) The method of claim 11 wherein the functional domain is a diagnostic or therapeutic functional domain.

13) The method of claim 12 wherein the target is a tissue, organ or cell and the therapeutic functional domain is toxic to the target tissue, organ or cell.

14) The method of claim 12 wherein the target is a tissue, organ or cell and the therapeutic functional domain stimulates the target tissue, organ or cell.

15) The method of claim 4 wherein the multifunctional polypeptide further comprises a functional domain.

16) A method of killing a tissue, organ or cell in a subject comprising administering to said subject a multifunctional polypeptide comprising a targeting domain, a first cytotoxic domain and a second cytotoxic domain, wherein said targeting domain specifically targets said polypeptide to said tissue, organ or cell and said first cytotoxic domain exerts its cytotoxic effect through a mechanism independent of the mechanism through which said second cytotoxic domain exerts its cytotoxic effect.

17) The method of claim 16 wherein said tissue, organ or cell is a diseased, injured or infected tissue, organ or cell.

18) The method of claim 17 wherein said diseased, injured or infected tissue, organ or cell is a cancerous tissue, organ or cell.

19) The method of claim 18 wherein said cancerous tissue, organ or cell is a tumor.
